(19) United States
(12) Patent Application Publication
(10) Pub. No.: US 2024/0026423 A1
(43) Pub. Date: Jan. 25, 2024

(54) Determining the Quantity and Quality of a DNA Library

(71) Applicant: Robert Bosch GmbH, Stuttgart (DE)

(72) Inventors: Anne Hoffmann, Herrenberg (DE); Christian Grumaz, Waiblingen (DE); Karin Lemuth, Herrliberg (CH)

(21) Appl. No.: 18/257,233

(22) PCT Filed: Dec. 7, 2021
(86) PCT No.: PCT/EP2021/084559

§ 371 Date: Aug. 14, 2023

(30) Foreign Application Priority Data
Dec. 17, 2020 (DE) 10 2020 216 120.8

(51) Int. Cl. C12Q 1/6818; C12Q 1/6806

(52) U.S. Cl. CPC C12Q 1/6818; C12Q 1/6806

(57) ABSTRACT

The disclosure relates to an adapter dimer-specific probe for detecting adapter dimers in a DNA library, the probe being labelled, particularly by a fluorophore, and being designed to bind specifically to a first adapter nucleotide sequence of a first adapter molecule and to a second adapter nucleotide sequence of a second adapter molecule.

The present invention relates to an adapter dimer-specific probe, a method for detecting adapter dimers in a deoxyribonucleic acid (DNA) library, and a cartridge designed so as to carry out the method according to the preamble of the independent claims.

### PRIOR ART

The publication by Hardigan et al. “CRISPR/Cas9-targeted Removal of Unwanted Sequences From small-RNA Sequencing Libraries. Nucleic Acids Research 2019” relates to the removal of adapter dimers from nucleic acid libraries. The removal is accomplished by a specific cleavage of the adapter dimers using the CRISPR/Cas9 system. In order to verify successful removal of the adapter dimers, a quantification of the adapter dimers by means of quantitative PCR is described. Adapter-overlapping primers are used.

The publication by Shore et al. “Small RNA Library Preparation Method for Next-Generation Sequencing Using Chemical Modifications to Prevent Adapter Dimer Formation. PLoS One 2016” relates to adapter molecules for creating sequencer nucleic acid libraries, avoiding the formation of adapter dimers.

WO 2014/144979 A1 discloses a reduction or inhibition of the formation of adapter dimers in the preparation of nucleic acid libraries.

WO 2019/222706 A1 relates to the sequencing of genomic DNA. Methods relating to polynucleotide libraries are provided.

WO 2009/106308 A2 describes the processing of nucleic acids for the creation of sequenceable nucleic acid libraries.

WO 2015/044412 A1 discloses adapter molecules for creating sequenceable nucleic acid libraries while avoiding the formation of adapter dimers.

Today, Next Generation Sequencing (NGS) methods are used nearly exclusively for sequencing DNA probes. The term NGS refers to a whole series of different sequencing methods, all of which are essentially characterized by the fact that a greater quantity of tests can be carried out at the same time automatically and in a short time. By means of NGS, it is possible, for example, to discover new disease-relevant genes and disease-causing genetic mutations, i.e. changes in the genetic material. Such insights help to understand basic biological processes and gain information about the development of genetic diseases.

One of the NGS methods is targeted sequencing (or amplicon sequencing), in which the fragment lengths of the DNA fragments to be sequenced are already known prior to the sequencing run due to corresponding sample preparation.

In the NGS, accurate quantification and quality analysis of the DNA library are carried out prior to each sequencing run in order to ensure a successful sequencing run. During the quantification, the concentration of the fragments is determined in the DNA library. For example, in the quality analysis, the fragment lengths of the DNA fragments to be sequenced are determined, as well as the formation of unwanted adapter dimers or other artifacts. Quantification and quality analysis are done independently of each other by means of various methods and technologies.

### DISCLOSURE OF THE INVENTION

According to the present invention, there is provided an adapter dimer-specific probe, a method for detecting adapter dimers in a DNA library, and a cartridge designed so as to carry out the method, having the characterizing features of the independent claims.

While the available NGS methods differ in the reagents and protocols to be used, the essential method steps are the same for all NGS methods. First, the DNA to be sequenced must be prepared for sequencing. For this purpose, the DNA sample to be sequenced is enzymatically or mechanically cut into small fragments. The fragment length of the DNA fragments depends on the respective method. Subsequently, short, artificially produced DNA pieces called adapter molecules are attached to the two free ends of the DNA fragments, usually by a ligation step. The adapter molecules are constructed so as to have a nucleotide sequence that is complementary to primers used. Primers are oligonucleotides with a known nucleotide sequence, which serve as a starting point for DNA-replicating enzymes such as DNA polymerase.

In order to provide a sufficient number of copies of DNA fragments for sequencing, the DNA fragments flanked by adapter molecules are duplicated, for example by polymerase chain reaction (PCR-based) methods. If the DNA is fragmented, equipped with adapter molecules, and specifically enriched, it is referred to as a DNA library (DNA library).

For example, the real-time quantitative PCR (qPCR) in which the amplified DNA fragments are quantified in real time during a PCR cycle is particularly suitable for quantifying the DNA library. For example, the quantification is done by fluorescence measurements. Fluorescent dyes are incorporated into the DNA fragments, thereby increasing the fluorescence proportionally with the amount of amplified DNA fragments.

In the manufacture of DNA libraries, the adapter molecules not only bind to the free ends of the DNA fragments to be amplified, but can also connect to one another in order to create adapter dimers. These are undesirable and distort the result in the quantification of the fragments in the DNA library as well as in later sequencing. In the quantification, the number of DNA fragments flanked by adapter molecules appears to be higher in the case of the presence of adapter dimers than it really is, because the adapter dimer fragments are also quantified.

Thus, in order to be able to assess the quantification of the fragments in a DNA library and to be able to control the development of artifacts such as adapter dimers, further methods for checking the quality must be carried out in addition to the qPCR.

According to the invention, an adapter dimer-specific probe for detecting adapter dimers, in particular in a DNA library, is disclosed.

The adapter dimer-specific probe comprises a label, on the basis of which it is detectable. This labeling is in particular a fluorophore, so that fluorescent radiation is detected when the fluorophore is stimulated with light of a particular wavelength. Alternatively, the labeling of the adapter dimer-specific probe is a chemiluminescent labeling, by means of which a light emission is detected as a result of a chemical reaction (chemiluminescence) or an enzymatic reaction (bioluminescence). Furthermore, alternatively, the labeling of the adapter dimer-specific probe is a radioactive labeling, in which the radioactive radiation is detected, or a redox-active component whose activity is detected by electrochemical methods.

For example, the adapter dimer-specific probe is a so-called Taqman probe, which comprises a signal quencher. For example, in the case of a fluorophore as a label, the signal quencher extinguishes the fluorescence signal of the fluorophore in that the fluorophore delivers at least a portion of its energy to the signal quencher when stimulated by a light source.

The quenching occurs only when the signal quencher and the fluorophore are in close spatial proximity to one another. In DNA amplification, in particular of the complementary DNA strand, for example by a polymerase, the probe covalently bonded to the DNA strand is degraded. As a result, the signal quencher and the fluorophore move away from one another, and an increasing fluorescence of the fluorophore can be measured.

The adapter dimer-specific probe is designed so as to specifically bond to a first adapter nucleotide sequence of a first adapter molecule and to a second adapter nucleotide sequence of a second adapter molecule.

What is advantageous with the adapter dimer-specific probe according to the invention is that adapter dimers are directly and specifically bonded by the adapter dimer-specific probe and can thus be quickly and easily detected. No further time-intensive and/or cost-intensive methods for the detection of adapter dimers are thus necessary.

Furthermore, a method for detecting adapter dimers in a DNA library during qPCR is disclosed, wherein the DNA library comprises DNA fragments flanked at both free ends by adapter molecules and adapter dimers.

The method comprises the following steps:
a) adding an adapter dimer-specific probe to a qPCR batch comprising the DNA library
b) determining the number of adapter dimers contained in the DNA library by means of the signal emitted by the adapter dimer-specific probe
c) calculating the actual number of DNA fragments in the DNA library, taking into account the determined number of adapter dimers.

In general, the qPCR is a very exact method for determining the concentration of DNA fragments in the DNA library, because only specifically adapter-bonded fragments are amplified and thus quantified. However, if adapter dimers have also formed, the primers also bind to them during the qPCR, so that the adapter dimers are also co-quantified. In this case, the measured concentration of DNA is higher than it actually is.

Thus, it is conventionally necessary, for accurate quantification, to determine the fragment lengths of the fragments contained in the DNA library via another method, for example gel electrophoresis or, if applicable, capillary gel electrophoresis, in order to be able to calculate the actual number of DNA fragments in the DNA library. The number of adapter dimers is subtracted from the number of DNA fragments.

Advantageously, in the method according to the invention, the number of adapter dimers in the DNA library are determined in real time directly during the qPCR by means of the adapter dimer-specific probe. Thus, no time-consuming and labor-intensive methods are necessary, such as determining the fragment lengths of the DNA fragments and adapter dimers in the DNA library, in particular when the fragment lengths of the DNA fragments are known, as is the case with a DNA library for targeted sequencing, for example. In case of known fragment lengths of the DNA fragments, the number of DNA fragments can be calculated from the corrected quantification without the qPCR being followed by further analysis methods and processes. Advantageously, by means of the probe according to the invention and the method according to the invention, work steps as well as temporal, financial, and material resources are thus saved.

The determined number of adapter dimers can also be a decisive quality feature for whether the DNA library can be used for a subsequent sequencing.

Further advantageous embodiments result from the sub-claims.

In a particularly advantageous embodiment of the method, at least one adapter-specific probe for a first adapter molecule is further added to the qPCR batch in step a), and
in step b), the number of the first adapter molecules contained is additionally determined by means of the signal emitted by the adapter-specific probe, and in step c), the calculation of the actual number of DNA fragments in the DNA library is carried out, taking into account the determined number of adapter dimers and the determined number of first adapter molecules.

Advantageously, both the number of adapter dimers in the DNA library is detected in real time directly during the qPCR by means of the adapter dimer-specific probe, and the number of first adapter molecules by means of the adapter-specific probe. The adapter-specific probe binds to first adapter molecules flanking DNA fragments as well as to first adapter molecules of adapter dimers. By means of the adapter-specific probe, the total number of first adapter molecules is thus detected. In order to calculate the actual number of DNA fragments in the DNA library, the number of detected adapter dimers is subtracted from the number of detected first adapter molecules. Thus, no time-consuming and labor-intensive methods are necessary in order to determine the actual number of DNA fragments, such as a determination of fragment lengths of the DNA fragments and adapter dimers in the DNA library.

Thus, during the qPCR, the fragments contained in the DNA library are quantified by the signal from the adapter-specific probe, and the quality of the DNA library is simultaneously determined by the signal from the adapter dimer-specific probe. This conserves work steps as well as temporal, financial, and material resources.

The determined number of adapter dimers can also be a decisive quality feature for whether the DNA library can be used for a subsequent sequencing.

In a further advantageous embodiment of the method, the amount of DNA to be used for a subsequent sequencing, in particular NGS sequencing, is determined based on the calculation of the actual number of DNA fragments in the DNA library.

Determining the exact DNA fragment count in the DNA library for DNA sequencing is paramount. If the DNA loading on the sequencer is too low, the full potential is not exploited, which is therefore uneconomic. Overloading has the consequence that the signals for sequencing can no longer be read correctly. Advantageously, the calculated actual number of DNA fragments in the DNA library can be used in order to calculate the optimal amount of DNA to be used, and thus an optimal loading of the sequencer is carried out.

Furthermore, the determined number of adapter dimers is decisive as to whether the DNA library can be used for sequencing. If there is too great a proportion of adapter dimers, it should not be used.

It is advantageous when the method is a targeted sequencing in which the length of the DNA fragments is known.

Advantageously, the length of the DNA fragments is known from the specific sample preparation. Depending on the qPCR batch, this parameter is required in order to be able to use the optimal amount of DNA for targeted sequencing via a calculation of the quantity and quality of the DNA library. Further methods, in particular for the fragment length determination of the DNA fragments, are thus omitted by the selection of a targeted sequencing, which saves time and cost as well as work steps.

The subject matter of the invention is furthermore a cartridge, in particular a microfluidic cartridge, for example as described in DE102016222072A1 or DE102016222075A1, wherein the cartridge is designed so as to carry out the method according to the invention.

The described method steps for detecting adapter dimers of a DNA library in real time during a qPCR or for detecting adapter dimers and first adapter molecules of a DNA library in real time during a qPCR are then all carried out within the cartridge, for example. A subsequent sequencing is also carried out within the cartridge, for example. For example, preparatory steps for the qPCR are also carried out within the cartridge.

In particular in a microfluidic system, a combined quantity and quality control is advantageous, as no further methods need to be implemented.

Alternatively, all described steps are carried out manually.

### BRIEF DESCRIPTION OF THE DRAWINGS

Embodiments of the present invention are illustrated in the drawings and further explained in the subsequent description. The figures show:

FIG. 1: the schematic representation of an adapter dimer-specific probe according to the invention for detecting adapter dimers,

FIG. 2: the schematic representation of a method according to the invention for detecting adapter dimers in a DNA library during a qPCR in a first embodiment,

FIG. 3: the schematic representation of the method according to the invention for detecting adapter dimers in a DNA library during a qPCR in a second embodiment, and

FIG. 4: the schematic diagram of a cartridge according to the invention, designed so as to carry out the method according to FIG. 3.

### EMBODIMENTS OF THE INVENTION

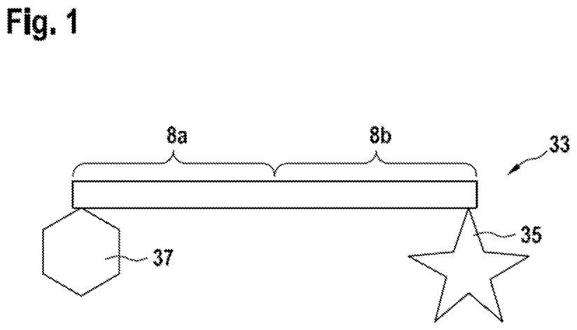

FIG. 1 shows an adapter dimer-specific probe 33 according to the invention for detecting adapter dimers 3, in particular in a DNA library.

The adapter dimer-specific probe 33 comprises a first DNA nucleotide sequence 8a, which is complementary to a portion of the adapter nucleotide sequence of a first adapter molecule 2a and a second DNA nucleotide sequence 8b, which is complementary to a portion of the adapter nucleotide sequence of a second adapter molecule 2b. By means of the first and second DNA nucleotide sequences 8a, 8b, the adapter dimer-specific probe 33 thus specifically binds to adapter dimers 3.

The adapter dimer-specific probe 33 comprises a fluorophore 35 as a label. If the fluorophore 35 is stimulated with light of a particular wavelength, it emits a detectable fluorescent radiation. In an alternative, non-illustrated embodiment, the labeling of the adapter dimer-specific probe 33 is a chemiluminescent labeling, a radioactive labeling, or a redox-active component.

For example, the adapter dimer-specific probe 33 is a Taqman probe comprising a signal quencher 37. The signal quencher 37 extinguishes the fluorescence signal of the fluorophore 35 in that the fluorophore 35 delivers at least a portion of its energy to the signal quencher 37 when stimulated by the light source.

The quenching occurs only when the signal quencher 37 and the fluorophore 35 are in spatial proximity to one another. In DNA amplification, for example by a DNA polymerase, the adapter dimer-specific probe 33 covalently bonded to the DNA strand is degraded. As a result, the signal quencher 37 and the fluorophore 35 move away from one another, and an increasing fluorescence of the fluorophore 35 can be measured.

In an alternative embodiment, the adapter dimer-specific probe 33 is a different type of probe.

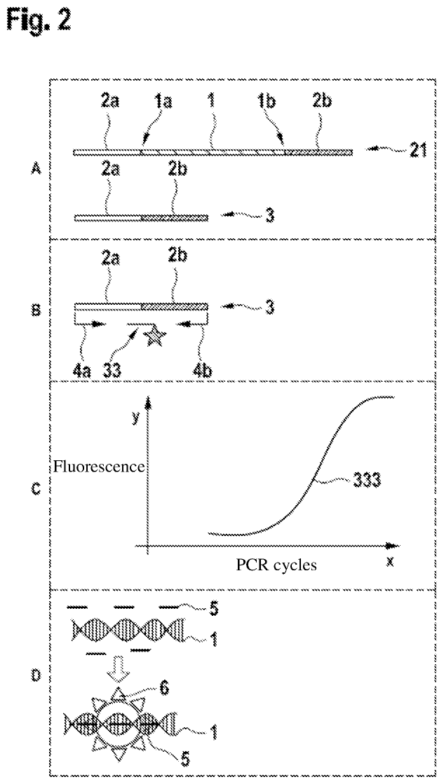

In FIG. 2, a method according to the present invention for detecting adapter dimers 3 in a DNA library during a qPCR is shown, in a first embodiment.

Firstly, and not shown in FIG. 2, a DNA sample is prepared such that DNA fragments 1 are present. For this purpose, the DNA sample is enzymatically or mechanically decomposed into DNA fragments 1 or alternatively, for example, enriched with certain DNA fragments 1 by means of a targeted PCR. The fragment lengths of DNA fragments 1 are dependent on the respective method.

Then, as shown in step A, first adapter molecules 2a and second adapter molecules 2b are added to the DNA fragments 1.

For example, the adapter molecules 2a, 2b are pseudo-double-stranded adapter molecules 2a, 2b and, for example, are brought asymmetrically through a ligation to the two free ends 1a, 1b of the DNA fragments 1. The first adapter molecules 2a thus specifically bind to the first free end 1a of the DNA fragments 1 and the second adapter molecules 2b thus specifically bind to the second free end 1b of the DNA fragments 1. In this way, DNA fragments 21 flanked by adapter molecules 2a, 2b are produced.

The first adapter molecule 2a has a nucleotide sequence that is complementary to a first primer. The second adapter molecule 2b has a nucleotide sequence that is complementary to a second primer.

The adapter molecules 2a, 2b not only bind to the free ends of the DNA fragments 1, but can also connect to one another, albeit less efficiently, so that adapter dimers 3 are produced. These are undesired and, among other things, distort the result of a later quantification of the DNA fragments 1.

In a further step, not shown in the figure, the flanked DNA fragments 21 and the adapter dimers 3 are duplicated, for example by a PCR method.

Hereafter, there is a DNA library comprising DNA fragments 1, flanked at both free ends by adapter molecules 2a, 2b, and adapter dimers 3.

In order to be able to determine the amount of DNA fragments 1 and adapter dimers 3, for example for analyses or for sequencing of the DNA fragments 1, a qPCR is carried out. In preparation for this and shown in step B, a first primer 4a and a second primer 4b are added to the batch, which serve as the starting point for DNA-replicating enzymes, for example DNA polymerase. The first primer 4a specifically binds to the complementary nucleotide sequence of the first adapter molecule 2a, and the second primer 4b specifically binds to the complementary nucleotide sequence of the second adapter molecule 2b. The nucleotide sequence complementary to the primers 4a, 4b of the adapter molecules 2a, 2b is, for example, only a subregion of the nucleotide sequence of the adapter molecules 2a, 2b at the respective unbonded ends of the adapter molecules 2a, 2b. Furthermore, an adapter dimer-specific probe 33, as described for example in FIG. 1, is added to the qPCR batch.

The adapter dimer-specific probe 33 specifically binds to a first adapter nucleotide sequence of a first adapter molecule 2a and to a second adapter nucleotide sequence of a second adapter molecule 2b. Thus, the adapter dimer-specific probe 33 is designed so as to specifically bind to adapter dimers 3.

In the subsequent qPCR, the flanked DNA fragments 21 as well as the adapter dimers 3 are duplicated. During the DNA amplification, after each cycle, the fluorescence emitted by the adapter dimer-specific probe 33 is measured, and an adapter dimer-specific fluorescence curve 333 is recorded as shown in step C. The x-axis shows the PCR cycles, and the y-axis shows the measured fluorescence. By means of a standard curve, a molecule number is assigned to the fluorescence signals. This allows the number of adapter dimers 3 to be calculated.

In addition, the amplified DNA fragments 1 are also quantified in real time after each PCR cycle. In step D above, a DNA fragment 1 is depicted, as well as a DNA intercalating label 5. The DNA intercalating label 5, for example a fluorescent dye, is incorporated into the DNA fragments 1 as shown in step 4 below, whereby the signal 6 of the label 5, in particular the fluorescent signal, increases proportionally with the amount of amplified DNA fragments 1. In addition, and not shown in the figures, the DNA-intercalating labeling 5 is also incorporated into the adapter dimers 3. Thus, the total concentration of DNA fragments 1 and adapter dimers 3 is detected here. A corrected quantification can be calculated from this by subtracting the concentration of adapter dimers 3 from the concentration of detected total DNA. If the fragment lengths of DNA fragments 1 are known, such as during a sample preparation for targeted sequencing, then the actual number of DNA fragments 1 can be calculated in the DNA library.

The determined number of adapter dimers 3 can also be a decisive quality feature for whether the DNA library can be used for a subsequent sequencing.

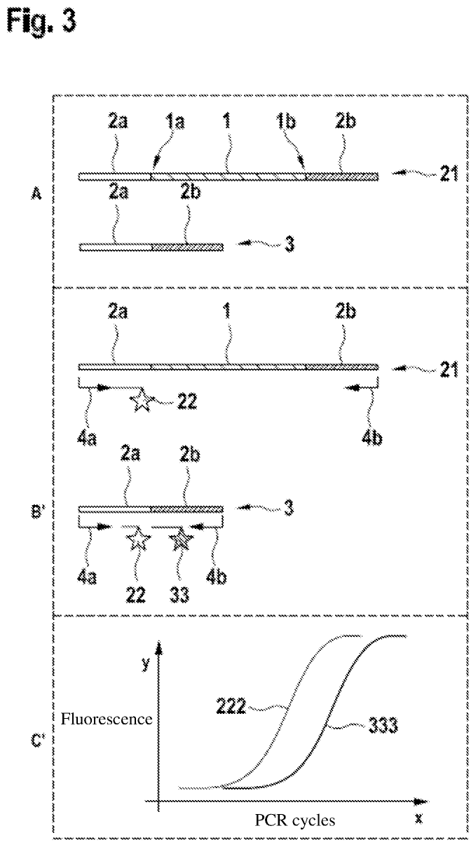

FIG. 3 shows a method according to the invention for detecting adapter dimers 3 in a DNA library during a qPCR in a second embodiment.

In contrast to the method described for FIG. 2 in the first embodiment, an adapter-specific probe 22 is also added to the qPCR batch in addition to the adapter dimer-specific probe 33 in step B′.

The adapter-specific probe 22 specifically binds to a first adapter nucleotide sequence of a first adapter molecule 2a. Thus, the adapter-specific probe 22

binds to a first adapter molecule 2a which flanks a DNA fragment 1 as well as to a first adapter molecule 2a of an adapter dimer 3. By means of the adapter-specific probe 22, the total number of first adapter molecules 2a is thus detectable, which is representative of the total number of flanked DNA fragments 21.

In the subsequent qPCR, the flanked DNA fragments 21 as well as the adapter dimers 3 are duplicated. During the DNA amplification, after each cycle, the fluorescence emitted by the adapter dimer-specific probe 33 is detected, and an adapter dimer-specific fluorescence curve 333 is recorded as shown in step C′.

Additionally, after each amplification cycle, the fluorescence emitted by the adapter-specific probe 22 is detected, and an adapter-specific fluorescence curve 222 is recorded as shown in step C′. The x-axis shows the respective PCR cycles, and the y-axis shows the measured fluorescence.

By means of a respective standard curve, each of the adapter dimer-related fluorescence signals and the adapter-related fluorescence signals is assigned a molecule number. This is done simultaneously to the recording of the fluorescence curves 222, 333. In order to then calculate the actual number of DNA fragments 1 in the DNA library, the determined number of adapter dimers 3 is subtracted from the number of determined first adapter molecules 2a.

Alternatively, and not shown in FIG. 3, the adapter-specific probe 22 specifically binds to an adapter nucleotide sequence of a second adapter molecule 2b.

For example, the actual number of DNA fragments 1 in the DNA library is relevant for determining the amount of DNA molecules to be used for a subsequent sequencing, in particular for NGS sequencing.

The determined number of adapter dimers 3 can also be a decisive quality feature for whether the DNA library can be used for a subsequent sequencing.

Depending on the number of different DNA fragments 1, and thus also different adapter molecules 2a, 2b used for example simultaneously in the methods described in FIGS. 2 and 3, different artifacts, in particular adapter dimers 3, can be formed. Of course, a plurality of different chimeric sequence-specific probes can then be used in the methods for detecting artifacts, such as the adapter dimer-specific probe 33 for detecting adapter dimers 3. A plurality of respective adapter-specific probes 22 can then be used in order to detect the adapter molecules 2a, 2b, for example.

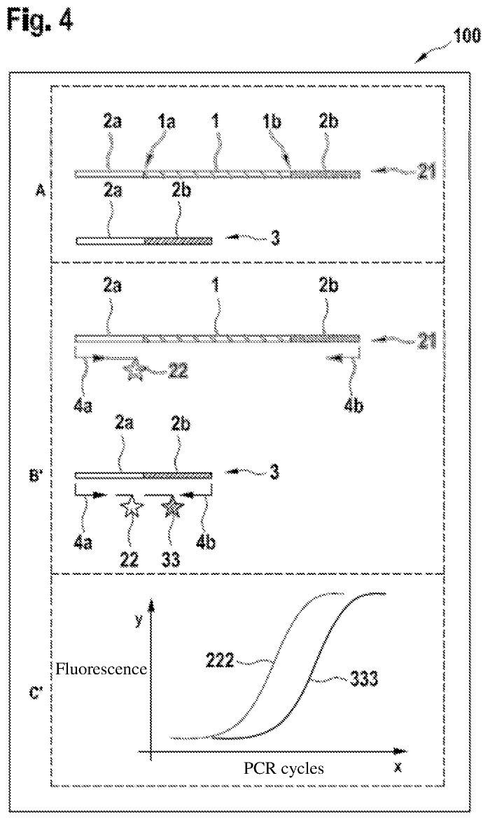

FIG. 4 shows a cartridge 100 according to the invention, in particular a microfluidic one. The cartridge 100 is designed so as to carry out a method according to the second embodiment, as described above in FIG. 3.

The steps described in FIG. 3 are then all carried out within the cartridge 100, for example. For example, a subsequent sequencing is also carried out within the cartridge 100.

Alternatively, and not shown in FIG. 4, the cartridge 100 is designed so as to carry out a method according to the first embodiment as described in FIG. 2, in particular with subsequent sequencing.

In an alternative embodiment, all described steps are carried out manually.

1. An adapter dimer-specific probe configured to detect adapter dimers in a DNA library, wherein comprising:
a fluorophore labelling the probe and designed to bind specifically to a first adapter nucleotide sequence of a first adapter molecule and to a second adapter nucleotide sequence of a second adapter molecule.

2. A method for detecting adapter dimers in a DNA library during a qPCR, wherein the DNA library comprises DNA fragments, which are flanked at both free ends by adapter molecules, and adapter dimers, comprising:
adding the adapter dimer-specific probe according to claim 1 to a qPCR batch comprising the DNA library;
determining a number of adapter dimers contained in the DNA library using a signal emitted by the adapter dimer-specific probe; and
calculating an actual number of DNA fragments in the DNA library using the determined number of adapter dimers.

3. The method according to claim 2, wherein:
adding the adapter dimer-specific probe includes adding at least one adapter-specific probe for a first adapter molecule;
determining the number of adapter dimers includes determining the number of the first adapter molecules contained using a signal emitted by the adapter-specific probe; and
calculating the actual number of DNA fragments includes calculating the actual number of DNA fragments using the determined number of first adapter molecules.

4. The method according to claim 2, further comprising:
determining the amount of DNA to be used in NGS sequencing based on the calculation of the actual number of DNA fragments in the DNA library.

5. The method according to claim 4, wherein it is a targeted sequencing in which a length of the DNA fragments is known.

6. A microfluidic cartridge, designed to carry out the method according to claim 2.
